(19) United States
(12) Patent Application Publication
(10) Pub. No.: US 2003/0088172 A1
(43) Pub. Date: May 8, 2003

(54) Magnetic resonance tomography apparatus with damping of mechanical vibrations by the use of material with electrostrictive properties

(76) Inventor: Rainer Kuth, Herzogenaurach (DE)

Correspondence Address: SCHIFF HARDIN & WAITE, 6600 SEARS TOWER, 233 S WACKER DR, CHICAGO, IL, 60606-6473, US

(21) Appl. No.: 10/256,376

(22) Filed: Sep. 27, 2002

(30) Foreign Application Priority Data
Sep. 27, 2001 (DE) 10147745.7

(51) Int. Cl. A61B 5/05

(52) U.S. Cl. 600/407

(57) ABSTRACT

In a magnetic resonance tomography apparatus noise suppression by strong damping of mechanical vibrations, in particular gradient coils and magnet vessels, is achieved through the use of composite materials that have electrostrictive properties. An MR tomography machine has a basic field magnet that is surrounded by a magnet casing that surrounds and delimits an interior space, a gradient coil system located in this interior space, and damping elements made from a material with an electrostrictive property are provided on an inner side, delimiting the interior space of the magnet casing for the purpose of absorbing acoustic vibrations that are produced upon switching of the gradient coil system.

### BACKGROUND OF THE INVENTION

1. Field of the Invention

The present invention relates in general to MR tomography as used in medicine for examining patients. The present invention relates, in particular, to an MR tomography apparatus wherein vibrations of apparatus components that negatively influence many aspects of the overall system are reduced.

2. Description of the Prior Art

Magnetic Resonance Tomography (MRT) is based on the physical phenomenon of nuclear spin resonance and has been used successfully as imaging method for over 15 years in medicine and in biophysics. In this method of examination, the object is exposed to a strong, constant magnetic field. This aligns the nuclear spins of the atoms in the object, which were previously oriented irregularly. Radio-frequency waves can now excite these “ordered” nuclear spins to a specific oscillation. In MRT, this oscillation generates the actual measuring signal that is picked up by means of suitable receiving coils. Owing to the use of inhomogeneous magnetic fields, generated by gradient coils, it is possible to code the measurement object spatially in all three spatial directions. The method permits a free choice of the layer to be imaged, as a result of which it is possible to obtain tomographic images of the human body in all directions. MR as a tomographic technique in medical diagnostics is distinguished first and foremost as a “non-invasive” method of examination by a versatile contrast capability. MRT has developed into a method far superior to x-ray computer tomography (CT) because of the excellent ability to display the soft tissue. Currently, MRT is based on the application of spin echo and gradient echo sequences that permit an excellent image quality with measuring times in the range of seconds to minutes.

Continuous technical development of the components of MR systems, and the introduction of high-speed imaging sequences, have opened up ever more fields of use for MR in medicine. Real time imaging for supporting minimally invasive surgery, functional imaging in neurology and perfusion measurement in cardiology are only a few examples.

The basic design of one of the central parts of such an NMR machine is illustrated in FIG. 4. It shows a superconducting basic field magnet 1 (for example an axial superconducting air-coil magnet with active stray field screening) which generates a homogeneous magnetic basic field in an interior space. The superconducting magnet 1 in the interior space has coils which are located in liquid helium. The basic field magnet is surrounded by a two-shell tank which is made from stainless steel, as a rule. The inner tank, which contains the liquid helium and serves in part also as a winding body for the magnet coils is suspended at the outer tank, which is at room temperature, via fiberglass-reinforced plastic rods which are poor conductors of heat. A vacuum prevails between the inner and outer tanks. The inner and outer tanks are referred to as a magnet vessel.

The cylindrical gradient coil system 2 in the interior space of the basic field magnet 1 is inserted concentrically into the interior of a support tube by means of support elements 7. The support tube is delimited externally by an outer shell 8, and internally by an inner shell 9. The function of the layer 10 will be explained later. The gradient coil system is powered by a power supply_.

The gradient coil system 2 has three component windings which respectfully generate gradient fields, each being proportional to the current in the coil, and which are spatially perpendicular to one another in each case. As illustrated in FIG. 5, the gradient coil system 2 includes an x coil 3, a y coil 4 and a z coil 5, which are respectively wound around the coil core 6 and thus respectively generate gradient fields in the directions of the Cartesian co-ordinates x, y and z. Each of these coils 3, 4 and 5 is provided with a dedicated power supply unit in order to generate independent current pulses with accurate amplitudes and timing in accordance with the sequence programmed in the pulse sequence controller. The required currents are at approximately 250 A.

Located inside the gradient coil is the radio-frequency resonator (RF coil or antenna; not illustrated in FIGS. 4 and 5). Its task is to convert the RF pulses output by a power transmitter into an alternating electromagnetic field for the purpose of exciting the atomic nuclei, and subsequently to convert the alternating field emanating from the preceding nuclear moments into a voltage supplied to the reception path.

Since the gradient switching times are to be as short as possible, current rise rates of the order of magnitude of 250 kA/s are necessary. In an exceptionally strong magnetic field as is generated by the basic field magnet 1 (typically between 0.22 and 1.5 tesla), such switching operations are associated with strong mechanical vibrations because of the Lorentz forces that occur in the process. All system components (housing, covers, tank of the basic field magnet and magnet casing, RF body coil etc.) are excited to forced vibrations.

Since the gradient coil is generally surrounded by conductive structures (for example magnet vessel made from stainless steel), the pulsed fields start in these eddy currents which exert force effects on these structures due to interaction with the basic magnetic field, and likewise excite these structures to vibrations.

These vibrations of the various MR apparatus components act negatively in many ways on the MR system:

1. Strong airborne noise is produced, which constitutes an annoyance to the patient, the operating staff and other persons in the vicinity of the MR system.

2. The vibrations of the gradient coil and of the basic field magnet, and their transmission to the RF resonator and the patient bed in the interior space of the basic field magnet and/or the gradient coil, are expressed in inadequate clinical image quality which can even lead to misdiagnosing (for example in the case of functional imaging, fMRI).

3. If the vibrations of the outer tank are transmitted to the inner tank via the GRP rods, or the superconductor itself is excited to vibrate, increased helium damping occurs—in a way similar to in an ultrasonic atomizer—in the interior of the tank, thus necessitating the subsequent supply of a larger quantity of liquid helium, and this entails higher costs.

4. High costs arise also due to the need for a vibration-damping system set-up—similar to an optical table—in order to prevent transmission of the vibrations to the ground, or vice versa.

In the prior art, the transmission of vibrational energy between the gradient coil and the further components of the tomography apparatus (magnet vessel, patient bed, etc.) is counteracted by the use of mechanical and/or electromechanical vibration dampers. The following methods are customarily used:

I) The vibrational energy is converted into heat through the use of passively acting vibration-absorbing materials (for example rubber bearings or viscous insulating materials). In particular, the noise production path over the interior of the MR apparatus, that is to say production of noise by vibration of the gradient coil and transmission of the noise to the support tube located in the gradient coil (8, 9 FIG. 2), which emits the noise inwardly to the patient and the interior space, is blocked in U.S. Pat. No. 4,954,781 by a damping viscoelastic layer 10 (FIG. 2) in the double-ply interior of the support tube. Furthermore, it is known to achieve the aforementioned blocking of the noise production path by inserting sound-absorbing so-called acoustic foams into the region between support tube and gradient coil.

II) Mechanical decoupling, for example by means of the support elements 7 illustrated in FIG. 2.

III) the use of vacuum or encapsulation of the vibration source by means of which the inner shell noted in FIG. 1 is decoupled from the outer shell of the vacuum tank.

IV) Specific stiffening of vibrationally affected structures, for example by using thick and heavy materials or by means of damping layers (for example tar) applied from “outside”.

V) Generally integrated magnetostrictors that experience an elastic change in shape in a changing magnetic field.

Nevertheless, the acoustic emission of a conventional MR apparatus continues to be very high.

### SUMMARY OF THE INVENTION

It is an the object of the present invention to reduce the noise transmission during operation of an MR apparatus.

This object is achieved according to the invention in an MR tomography machine has a basic field magnet surrounded by a magnet casing that surrounds and delimits an inner space, a gradient coil system fastened in the interior space of the magnet via support elements, and a radio-frequency resonator also arranged in the interior space, and electrostrictive damping elements disposed between at least two concentric layers for absorbing acoustic vibrations that are produced upon switching of the gradient coil system.

The damping elements are comprised of a material that is doped with electrostrictive liquid crystal elastomers.

In this case, the doped material constitutes an elastomeric or rubber-like substance.

The property of electrostriction is manifest by a mechanical deformation, that is to say a change in length, of a material—in general of an insulator—when the electric field in which it is located is changed. The inverse effect is the piezoelectric effect, in which an electric polarization, that is to say a change in voltage, occurs when an appropriate material is deformed.

There are various fields of use and/or possibilities of arrangement for the damping elements according to the invention:

arrangement of the damping elements between the gradient coil system and the magnet casing,

arrangement of the damping elements between the gradient coil system and the radio-frequency resonator,

arrangement of the damping elements between the magnet casing and the bottom,

implementing further damping elements made from a material with an electrostrictive property in the gradient coil.

The damping elements can be advantageously constructed as plates, rings or ring segments etc., or as a thin layer.

Furthermore according to the invention, a damping laminated sheet structure is provided on an inner side, delimiting the inner space, of the magnet casing, that has at least two sheets with damping elements respectively located therebetween.

The possibility exists in this case that the damping laminated sheet structure constitutes an open system in which an inner sheet forms the vacuum-bearing inner wall of the magnet casing, and an outer sheet forms a damping outer wall of the magnet casing with the damping element situated between the two sheets.

In some circumstances, this open system extends only over the partial surface of the magnet casing that faces the interior space.

Another design possibility is that the damping laminated sheet structure constitutes a closed system in which both the inner sheet and the outer sheet form the vacuum-bearing wall of the magnet casing, and a damping element is located between the two sheets.

It is possible in this case that the closed system extends only over the partial surface of the magnet casing that faces the interior space, or else over the entire surface of the magnet casing.

It can equally be advantageous when the damping laminated sheet structure in a multilayer design forms a closed system composed of a number of sheets with the damping elements situated therebetween.

The energy for driving the electrostrictive damping elements can be drawn from the power supply for the gradient coils.

The electrostrictive damping elements can be controlled according to the invention by a trainable electronic system.

Also according to the invention is the use of an electrostrictive material for damping vibrations in an MR tomography apparatus that has a basic field magnet surrounded by a magnet casing that surrounds and delimits an interior space, a gradient coil system suspended concentrically in this interior space via support elements, and a radiator frequency transmitter suspended concentrically in the interior space and electrostrictive damping elements between at least two concentric layers damping elements for absorbing acoustic vibrations that are produced upon switching over of the gradient coil system.

The material of which the damping elements is doped with electrostrictive liquid crystal elastomers.

An advantageous type of use of this material can be the use of an elastomeric or rubber-like substance as the doped material.

### DESCRIPTION OF THE DRAWINGS

FIG. 1 is a schematic section through the basic field magnet of a magnetic resonance apparatus showing the components of the interior space thereof.

FIG. 1a is a section through the inventive damping laminated sheet structure which constitutes an open system.

FIG. 1b is a section through the inventive damping laminated sheet structure which represents a closed system which extends only over the partial surface of the magnet casing which faces the interior space.

FIG. 1c is a section through the inventive damping laminated sheet structure which represents a closed system which extends over the entire surface of the magnet casing.

FIG. 1d is a section through the inventive damping laminated sheet structure which forms a closed system composed of a number of sheets having damping planes located therebetween.

FIG. 2a is a section through the magnet casing at the end face, with use being made of radially arranged stiffeners.

FIG. 2b is a front view of the end face of the basic field magnet, with use being made of radially arranged stiffeners.

FIG. 3 shows the patient couch, the vibrations of which are damped by integrating inventive damping layers into the support structure.

FIG. 4 is a perspective illustration of the basic field magnet of the apparatus of FIG. 1.

FIG. 5 is a perspective illustration of the gradient coil with three component windings of the apparatus of FIG. 1.

### DESCRIPTION OF THE PREFERRED EMBODIMENTS

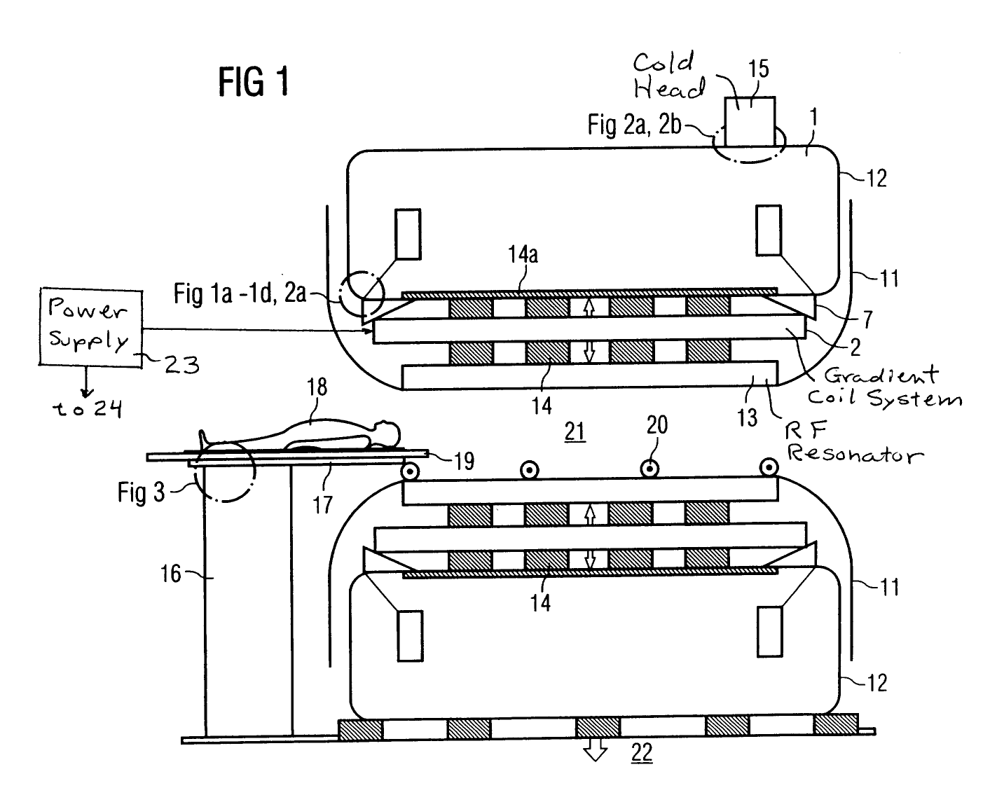

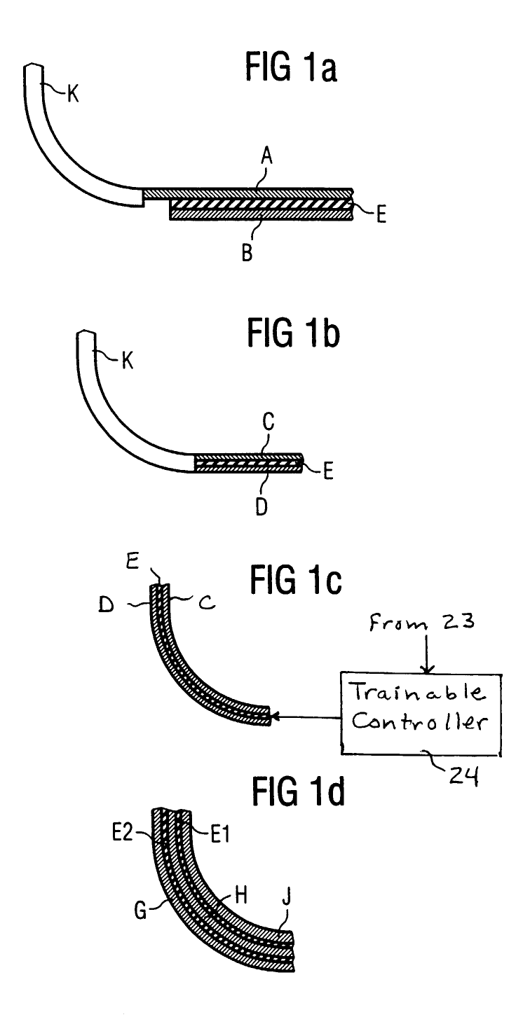

FIG. 1 is a schematic section through the basic field magnet 1 of an MR apparatus and through further components of the interior space which the magnet encloses. The basic field magnet 1 includes superconducting magnet coils which are located in liquid helium, and is surrounded by a magnet casing 12 in the form of a double-shell tank. The so-called cold head 15 fitted outside on the magnet casing 12 is responsible for keeping the temperature constant. The gradient coil 2 is suspended concentrically via support elements 7 in the inner space surrounded by the magnet casing 12 (also termed magnet vessel). The radio-frequency resonator 13 is likewise inserted concentrically, in turn, in the interior of the gradient coil 2. The function of the resonator 13 is to convert RF pulses output by a power transmitter into an alternating magnetic field for the purpose of exciting atomic nuclei of a patient 18, and subsequently to convert the alternating field emanating from the precessing nuclear moments into a voltage fed to the reception path. On a patient couch 19, which is located on a slide rail 17, the patient 18 is moved via rollers 20 fitted on the RF resonator 13 into the opening and the interior space of the system. The slide rail 17 is mounted on a vertically adjustable supporting frame 16. FIG. 1 also shows, as examples, cowlings 11, and the floor 22 on which the MR apparatus stands.

The system illustrated diagrammatically in FIG. 1 has two sources of vibration or vibration centers. These are the cold head 15 and, the gradient coil 2.

The present invention permits the transmission of noise to be reduced at specific strategic points by the use of specific damping elements 14 or damping layers E.

The strategic points addressed, at which the damping elements 14 are to be used, are the interfaces between the gradient coil 2 and the magnet vessel 12, in particular the region of the magnet inner side 14 (warm bore) which is particularly sensitive to vibration, the region around the cold head 15, the patient couch 16, 17, 19, and between the magnet vessel 12 and the floor 22, as well as between the radio-frequency resonator 13 and the gradient coil 2.

A controlled mechanical damping is implemented in accordance with the invention between the gradient coil 2 and the magnet vessel 12 and between the magnet vessel 12 and the bottom 13, as well as between the radio-frequency resonator 13 and the gradient coil 2 by using materials that have electrostrictive properties.

Electrostrictive materials occurring in nature which exhibit a deformation produced by an electric field (the deformation being a quadratic function of the field strength), are crystals with one polar axis or a number of polar xes, for example quartz (SiO₂), tourmaline, barium titanate, and Seignette salt. So-called electrostriction materials, however, also can be produced artificially, for example by sintering selected ceramics (perovskites). The latter exhibit changes in length of 1 per thousand at approximately 2 kV/mm.

A notably larger tensile force is achieved with electrostriction of liquid crystal molecules (mesogenes) that are incorporated into elastomers. Although liquid crystal molecules can be easily aligned in an electric field, they behave like a liquid, that is they can neither withstand nor exert a mechanical tensile force. In order to prevent them from flowing, they have been incorporated into an elastomer. Elastomers such as rubber consist of polymers that form a 3-dimensional network, for which reason the polymer chains cannot slide on one another under deformation. The very dimensional stability of an elastomer doped with mesogenes stabilizes the order, but leaves the mesogenes enough space for the electrically induced alignment.

Because of its stable functional principle, the present invention is based on the recognition that such damping material is particularly well suited for use in MR systems, in particular in gradient coils and magnet vessels. Its very high damping effect—an ultrathin (<100 nm) liquid crystal elastomer film has a tensile force of 4% at only 1.5 MV/m—permits an efficient suppression of the mechanical vibrations and thereby contributes to the suppression of the undesirable noise production and/or noise transmission.

It is likewise within the invention to use this material to damp the vibrations within the gradient coil 2 itself. In this case, the material is arranged the location of the antinodes of the vibrations in order to reduce the amplitude of vibration.

Various designs can be implemented according to the invention:

FIG. 1a shows a system having two layers, disposed only at the inner side 14, delimiting the interior space 21, of the magnet casing 12. Like the end face K, the inner layer A has the function of maintaining the vacuum in the interior of the magnet casing 12 against the air pressure prevailing outside. This requires an adequate mechanical stiffness in order to withstand the static underpressure load. In the system illustrated in FIG. 1a, only the inner side 14, delimiting the interior space 21, of the magnet casing 12 is provided with a further sheet lamination B. This need not be vacuum-tight. Its purpose is to increase the stiffness and the damping of the inner side 14. The actual damping is effected, however, by a damping layer E which is illustrated between the two sheet laminations A and B as middle layer E. This is bonded to the adjacent metal layers A and B.

A deformation of the layer A caused, for example, by inductive forces that are produced by the switching of the gradient system, can be counteracted by changing a voltage applied to the layer E.

Since the outer layer B in FIG. 1a has no bearing function, the illustrated structure of the magnet casing 12 is designated as an open system.

By contrast, FIG. 1b shows a closed system. Here, the inner side 14, delimiting the interior space 21, of the magnet casing 12 likewise has an inner layer C and an outer layer D. Likewise located between the two layers is a damping layer E. The difference from the open system in FIG. 1a is, however, that together with the inner layer C the outer layer D must also, like the end face K, withstand the ultrahigh vacuum in the interior of the magnet casing 12. The two layers or sheets C and D therefore are welded to one another and to the shell K and thereby form a closed structural unit in the form of a sandwich design. This closed system is certainly more costly, but fundamentally has a higher degree of stiffness. Consequently, less of a demand is placed in this exemplary arrangement on the change in length and/or thickness of the electrostrictive layer E.

The sheet thicknesses of the respective layers can be the same in both systems, or different. In the embodiments of FIGS. 1a and 1b, a layered design with an electrostrictive intermediate layer exclusively in the region of the warm bore 14, that is particularly sensitive to vibration, (FIG. 1) is illustrated. A damping laminated sheet structure over the entire magnet casing 12 is also equally conceivable, as illustrated in FIG. 1c.

Damping which is certainly more expensive but also more effective is achieved in a layered design with more than two sheet laminations as in FIG. 1d, for example three sheet laminations G, H, J.

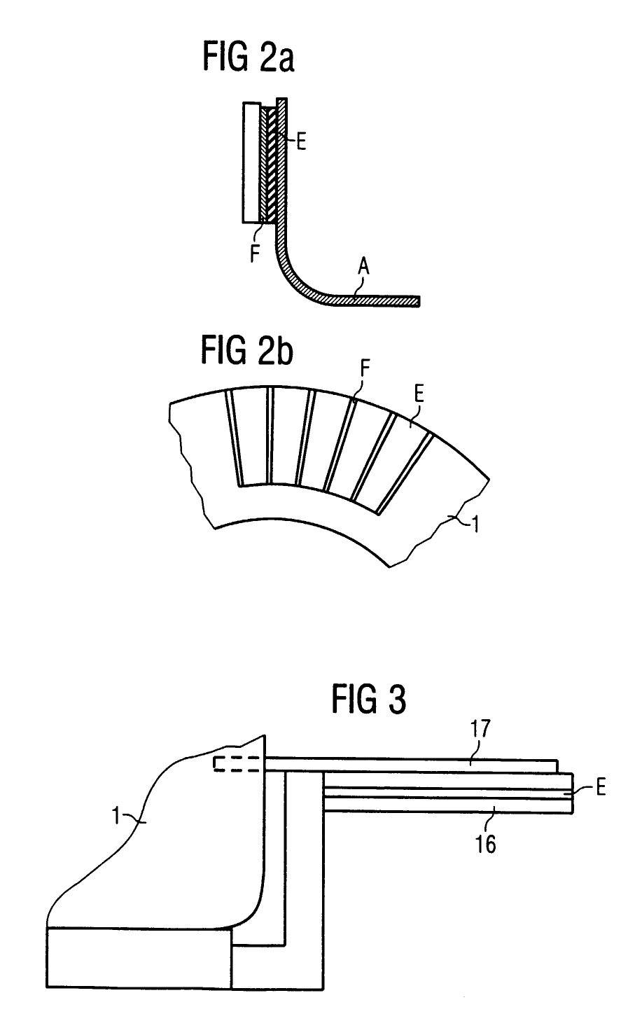

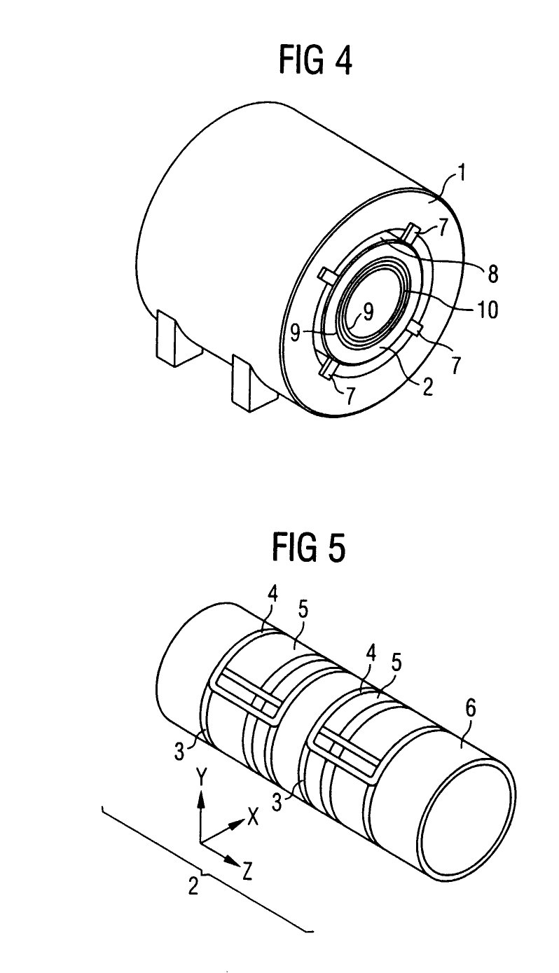

As mentioned above, a multilayer design increases the effectiveness of a counteractive control on the basis of a number of electrostrictive layers with reference to the overall surface. A still higher stiffness is obtained at the end face of the magnet casing 12, in particular, by fitting additional radially arranged stiffeners F (FIG. 2a, sectional view and FIG. 2b, front view). The damping layers E can be activated individually or collectively.

The design alternatives just set forth are suitable for preventing the spread of vibrations in the case of suitably adapted integration, specifically by annular isolation of the source of vibration, as is illustrated by the cold head 15, for example.

A patient couch is illustrated in FIG. 3. A trough-shaped board 19 on which the patient lies is mounted on a slide rail 17. The slide rail 17, itself horizontally movable, is located on a vertically adjustable supporting frame 16 by means of which the couch can be brought with the patient to the level of the roller bearings 20 and can be moved into the opening of the system.

Transmission of the vibrations of the magnet and/or the RF resonator to the patient couch 16, 17, 19 likewise can be prevented by integrating damping layers E into the support structure of the couch, that is to say into the board 19 and the slide rail 17 or between two parts, such as between the supporting frame 16 and slide rail 17, as well as by a damping roller bearing 20.

The energy for applying a voltage to the electrostrictive layer or for a change in voltage can be obtained, for example, via a transformer from the power supply 23 for the gradient coil system 2, as schematically indicated in FIG. 1c.

The electrostrictive damping elements or damping layers can be driven by a trainable electronic controller 24, as also schematically shown in FIG. 1c. This controller 24 controls the vibration-affected regions to a state minimum noise after the appropriate reaction time or dead time.

Although modifications and changes may be suggested by those skilled in the art, it is the intention of the inventor to embody within the patent warranted hereon all changes and modifications as reasonably and properly come within the scope of his contribution to the art.

I claim as my invention:

1. A magnetic resonance tomography apparatus comprising:
a basic field magnet surrounded by a magnet casing which surrounds and delimits an interior space;
a gradient coil system mounted in said interior space via support elements;
a radio-frequency resonator also disposed in said interior space; and
a plurality of damping elements disposed at selected locations for absorbing acoustic vibrations produced during switching of said gradient coil system, said damping elements containing a material having a property of electrostriction.

2. A magnetic resonance tomography apparatus as claimed in claim 1 wherein said damping elements comprise a material doped with electrostrictive liquid crystal elastomers.

3. A magnetic resonance tomography apparatus as claimed in claim 2 wherein said material comprises an elastomeric substance.

4. A magnetic resonance tomography apparatus as claimed in claim 1 wherein said damping elements are disposed between said gradient coil system and said magnet casing.

5. A magnetic resonance tomography apparatus as claimed in claim 1 wherein said damping elements are disposed between said gradient coil system and said radio-frequency resonator.

6. A magnetic resonance tomography apparatus as claimed in claim 1 wherein said damping elements are disposed between said magnet casing and a floor disposed beneath said magnet casing.

7. A magnetic resonance tomography apparatus as claimed in claim 1 comprising further damping elements, associated with said gradient coil, comprised of a material having an electrostrictive property.

8. A magnetic resonance tomography apparatus as claimed in claim 1 wherein said damping elements are selected from the group consisting of plates, rings, ring segments, and thin layers.

9. A magnetic resonance tomography apparatus as claimed in claim 1 wherein said magnet casing has an inner side, delimiting said interior space, and wherein said damping elements include a damping laminated sheet structure disposed on said inner side, and comprising at least two sheets with damping elements disposed between said two sheets.

10. A magnetic resonance tomography apparatus as claimed in claim 9 wherein said damping laminated sheet structure is an open system having an inner sheet forming a vacuum-bearing inner wall of said magnet casing, and an outer sheet forming a damping outer wall of said magnet casing, with a damping element disposed between said inner sheet and said outer sheet.

11. A magnetic resonance tomography apparatus as claimed in claim 10 wherein said open system extends only over a portion of a surface of said inner side.

12. A magnetic resonance tomography apparatus as claimed in claim 9 wherein said damping laminated sheet structure is a closed system having an inner sheet and an outer sheet both forming a vacuum-bearing wall of said magnet casing, and a damping element disposed between inner sheet and said outer sheet.

13. A magnetic resonance tomography apparatus as claimed in claim 12 wherein said closed system extends only over a portion of a surface of said inner side.

14. A magnetic resonance tomography apparatus as claimed in claim 12 wherein said closed system extends over an entirety of a surface of said inner side.

15. A magnetic resonance tomography apparatus as claimed in claim 1 wherein said damping elements are a part of a damping laminated sheet structure formed by two sheets with a damping element disposed between said two sheets.

16. A magnetic resonance tomography apparatus as claimed in claim 15 wherein said damping laminated sheet structure is a closed system comprising a plurality of sheets with damping elements disposed therebetween.

17. A magnetic resonance tomography apparatus as claimed in claim 1 further comprising a power supply for supplying power to said gradient coil system, and wherein energy for operating said damping elements is tapped from said power supply.

18. A magnetic resonance tomography apparatus as claimed in claim 1 comprising a trainable electronic system connected to said damping elements for controlling said damping elements.
